(19) United States
(12) Patent Application Publication
(10) Pub. No.: US 2005/0043807 A1
(43) Pub. Date: Feb. 24, 2005

(54) Two-thirds prosthetic arthroplasty

(76) Inventor: David John Wood, Perth (AU)

Correspondence Address: MICHAEL MOLINS;MOLINS & CO., SUITE S, LEVEL 6, 139 MACQUARIE ST, SYDNEY NSW, 2000, AU

(21) Appl. No.: 10/920,217

(22) Filed: Aug. 18, 2004

(30) Foreign Application Priority Data
Aug. 18, 2003 (AU) 2003904379

(51) Int. Cl. A61F 2/38

(52) U.S. Cl. 623/20.14; 623/20.15; 623/20.3

(57) ABSTRACT

A two-thirds prosthetic arthroplasty having a trochlear groove, a patello-femoral component, and either a lateral condyle or a medial condyle. The arthroplasty may be configured to be a prosthetic for either a right or left knee. The condyle and patello-femoral component are separated by a modified intercondylar notch that is blended to avoid protrusions or sharp angles. The prosthetic arthroplasty is inserted after preoperative MRI mapping of articular cartilage damage. The prosthetic arthroplasty may be cemented during surgery and the two-thirds design of the present invention obviates the problem of cement retrieval from remote parts of the prosthesis.

### BACKGROUND OF THE INVENTION

1. Field of the Invention

The invention pertains to prosthetic knees and more particularly to a two-thirds prosthetic knee, having a single condyle in a prosthetic patello-femoral joint.

2. Description of Related Art

The distribution of articular cartilage damage in the osteoarthritic knee is variable. It may involve one, two or all three compartments of the knee. Although unicompartmental tibio-femoral and unicompartmental patello-femoral partial knee replacements are available, combinations of two compartment replacements are not.

With increasing pressure towards minimally invasive surgery the concept of only replacing the diseased compartments is attractive. Accordingly there is a need for combinations of either the patellofemoral and medial tibiofemoral joint or patellofemoral and lateral patelofemoral joints respectively.

### SUMMARY AND OBJECTS OF THE PRESENT INVENTION

It is an object of the invention to provide a device comprising a prosthetic femoral component having a single condyle and a trochlear groove for the patella.

Methods of surgical implantation of this device are disclosed.

### BRIEF DESCRIPTION OF THE DRAWINGS

For a more complete understanding of the present invention and for further advantages thereof, reference is now made to the following Description of the Preferred Embodiments taken in conjunction with the accompanying Drawings in which:

FIG. 1 is a top plan view of a prosthetic femoral component.

FIG. 2 is a front elevation of a two-thirds prosthetic femoral component having a lateral condyle only.

FIG. 3 is a front elevation of a two-thirds prosthetic femoral component having a medial condyle only.

### DESCRIPTION OF THE PREFERRED EMBODIMENTS

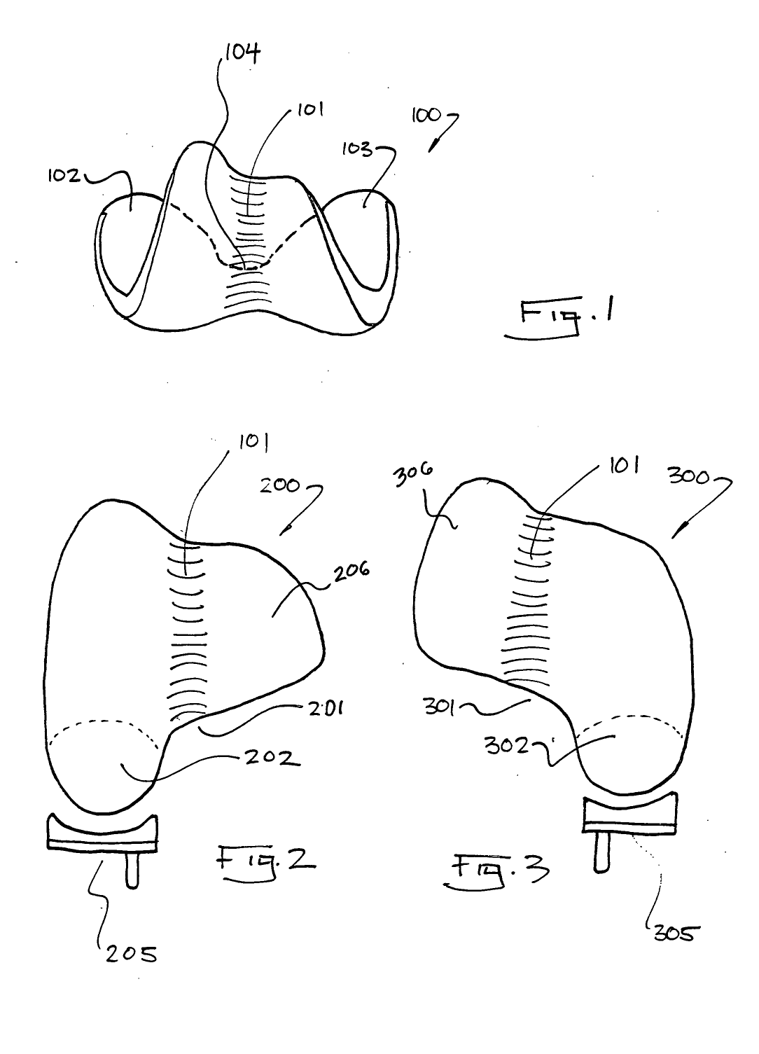

As shown in FIG. 1, a typical prosthetic right femoral component 100 comprises a curved and polished metal body having prosthetic equivalents of a trochlear groove 101 as well as femoral condyles, these being the lateral condyle 102 and the medial condyle 103. The intercondylar notch 104 defines a gap between the condyles.

The present invention is depicted by way of example in FIGS. 2 and 3. FIG. 2 illustrates a two-thirds prosthetic right femoral component 200 having a lateral condyle 202 only. The medial condyle is replaced by a patello-femoral joint component 206. FIG. 3 depicts a two-thirds prosthetic right femoral component 300 having a medial condyle 302 only. The lateral condyle is replaced by a patello-femoral joint component 306. The trochlear groove is preserved but note that the area of the intercondylar notch 201, 301 is blended to avoid protrusions or sharp angles. A typical fibial component 205, 305 is shown.

Aspects of the invention are based on the premise that there are distinct benefits obtained if only diseased compartments are replaced. A patient that receives a prosthetic of the type depicted in FIGS. 2 and 3 is retaining one natural femoral condyle. The remaining natural condyle and other natural tissue provide a useful source of orientation data which makes alignment of the two thirds prosthetic more likely to succeed. Further, because the two thirds prosthetic itself is smaller than the prior art type depicted in FIG. 1, a minimally invasive surgical approach or less invasive approach may be considered or actually implemented.

The principle of closely matching surgery to preoperative MRI mapping of articular cartilage damage is a principle of surgical planning for the two-thirds knee.

MRI evaluation cannot only evaluate the distribution of cartilage damage, but also size match the native tibial and femoral components to prosthetic implants. The femoral and tibial orientation, in terms of rotation and joint line level and slope and the orientation of the patellar centrum can be calculated using CT scanning. Following thorough clinical evaluation imaging and preoperative planning, a minimally invasive surgical approach may be considered and the smaller size of both femoral and tibial components will facilitate this. An antero-medial or antero-lateral approach may be used to match the tibio-femoral compartment to be replaced.

Following incision and capsulotomy some ligament balancing may be done before the tibial cut. For example the deep medial collateral ligament may be released if the medial compartment is to be replaced or the ilio-tibial band if the lateral compartment is to be replaced. The tibial cut should preferably be done first with standard extramedullary jigging or by computer assisted navigation if preferred. This will facilitate the first, distal femoral cut. Bone from the two aforementioned cuts is removed to permit closed cuts or reaming with an angled guarded saw or reamer over customized jigs. With bone removed following the single tibial and femoral cuts there is more space to perform an accurate patellar preparation.

Although the components may be cemented or uncemented, it is preferred that cement be used. The two-thirds knee obviates the problem of cement retrieval from remote parts of the prosthesis, a current problem in minimally invasive total knee replacement.

Care must be exercised to ensure a smooth transition between the retained femoral condyle and the prosthesis. Kinematic evaluation may be done following implantation.

While the present invention has been described with reference to particular details of construction, methods and procedures, these should be understood to be provided by way of teaching examples and not as limitations to the scope or spirit of the invention.

1. A two-thirds prosthetic arthroplasty comprising:
a condyle,
a patello-femoral joint component, and
a trochlear groove.

2. The prosthetic arthroplasty of claim 1 wherein:
the condyle is a lateral condyle.

3. The prosthetic arthroplasty of claim 1 wherein:
the condyle is a medial condyle.

4. The prosthetic arthroplasty of claim 1 wherein:
the condyle, the trochlear groove, and the patello-femoral joint component are arranged to form a prosthetic device for a right knee.

5. The prosthetic arthroplasty of claim 1 wherein:
the condyle, the trochlear groove, and the patello-femoral joint component are arranged to form a prosthetic device for a left knee.

6. The prosthetic arthroplasty of claim 1 wherein:
the condyle and the patello-femoral joint component are separated by a modified intercondylar notch that is blended to avoid protrusions and sharp angles.

7. A method for prosthetic knee replacement surgery, comprising the steps of:
1) preoperative MRI mapping of articular cartilage damage,
2) MRI evaluation to evaluate the distribution of cartilage damage and size matching of the native tibial and femoral components to prosthetic implants,
3) surgically implanting a two-thirds prosthetic arthroplasty having trochlear groove, a patello-femoral component, and a condyle.

8. The method of claim 7 wherein:
the condyle for the prosthetic arthroplasty is a medial condyle.

9. The method of claim 7 wherein:
the condyle for the prosthetic arthroplasty is a lateral condyle.

10. The method of claim 7 wherein:
the condyle and patello-femoral component are separate by a modified intercondylar notch is blended to avoid protrusions and sharp angles.

11. The method of claim 7, wherein:
an antero-medial approach is used when the prosthetic arthroplasty has a medial condyle.

12. The method of claim 7 wherein:
an antero-lateral approach is used when the prosthetic arthroplasty has a lateral condyle.

13. The method of claim 7 wherein:
the prosthetic arthroplasty is cemented in place.
